(19) United States
(12) Patent Application Publication
(10) Pub. No.: US 2025/0018041 A1
(43) Pub. Date: Jan. 16, 2025

(54) Use of Plasma-Treated Liquids to Treat Herpes Keratitis

(71) Applicant: Drexel University, Philadelphia, PA (US)

(72) Inventors: Jane Azizkhan-Clifford, Newtown, PA (US); Oleg Alekseev, Philadelphia, PA (US); Kelly Rose Donovan, Philadelphia, PA (US)

(21) Appl. No.: 18/768,359

(22) Filed: Jul. 10, 2024

Related U.S. Application Data
(63) Continuation of application No. 17/186,098, filed on Feb. 26, 2021, now abandoned.
Application No. 17/186,098 is a continuation of application No. 15/024,051, filed on Mar. 23, 2016, now abandoned.
(60) Provisional application No. 61/883,392, filed on Sep. 27, 2013.

(51) Int. Cl. A61K 41/00; A61K 9/00; A61K 9/08

(52) U.S. Cl. CPC A61K 41/0023; A61K 9/0048; A61K 9/08

(57) ABSTRACT

The present invention is directed toward the use of non-thermal plasma-treated liquids as treatment options for herpes keratitis.

### CROSS REFERENCE TO RELATED APPLICATIONS

This application is a continuation of U.S. patent application Ser. No. 17/186,098, filed Feb. 26, 2021; which is a continuation of U.S. patent application Ser. No. 15/024,051, filed Mar. 23, 2016; which is a National Stage Application filed under 35 U.S.C. 371 of International Application No. PCT/US2014/056491, filed Sep. 19, 2014; which claims priority to and the benefit of U.S. Patent Application Ser. No. 61/883,392, filed Sep. 27, 2013. All foregoing applications are incorporated herein by reference in their entireties for any and all purposes.

### SEQUENCE LISTING

The instant application contains a Sequence Listing which is being submitted herewith electronically in XML format and is hereby incorporated by reference in its entirety. Said XML copy, created on Sep. 25, 2024, is named 2024 Sep. 25-101311000187_Sequence Listing.xml and is 14,085 bytes in size.

### TECHNICAL FIELD

Aspects of the disclosed subject matter are in the field of treating herpes keratitis.

### BACKGROUND

Herpes keratitis is the leading cause of cornea-derived and infection-derived blindness in the developed world. HSV-1 and, to a lesser extent, HSV-2 are known to be the leading causes of virus-induced blindness in the Western world, with approximately 400,000 patients in the US currently afflicted with this disease, and 20,000 new cases appearing each year. More than 60% of the U.S. population aged 12 years and higher is positive for HSV-1, HSV-2, or both. Worldwide, 60% to 90% of the adult population is HSV-1 antibody positive. In one study, 100% of individuals older than 60 years were found to be HSV-1 seropositive. Despite the prevalence of HSV infections, however, only a small number of latently infected humans experience symptomatic disease. However, the disease is extremely painful. Approximately 25% of cases become the more severe stromal keratitis. Current treatment regimens include the use of antivirals, which must be administered as often as 9 times per day, severely impacting the quality of life. Drug resistance in the immunocompromised population is exceeding 10%, necessitating new therapies. There are many refractory cases of HK despite current antivirals and prevalence is 14-fold higher in the cornea-transplant population.

### SUMMARY

The present invention is directed toward the use of plasma-treated liquids as treatment options for herpes keratitis.

### BRIEF DESCRIPTION OF THE DRAWINGS

Aspects of the present subject matter will become apparent from the following detailed description of the subject matter when considered in conjunction with the accompanying drawings. For the purpose of illustrating the subject matter, there is shown in the drawings embodiments that are presently preferred, it being understood, however, that the subject matter is not limited to the specific descriptions disclosed. The drawings are not necessarily drawn to scale. In the drawings:

FIG. 1 is a photographic image of an eye afflicted with herpes keratitis.

FIGS. 2A-C describe the preparation and use of DBD plasma-treated liquids. FIG. 2A shows an experimental set-up for generation of non-thermal DBD plasma. Fully insulated electrode receives 15-to 20-kV current alternating at 1.0-KHz frequency. One milliliter liquid is placed into a custom-made glass holder (measurements are shown in inches) (FIG. 2B), such that there is a 1-mm gap between the insulated electrode and the liquid surface. The alternating current ionizes air molecules in the 1-mm gap, producing a characteristic purple glow (inset in FIG. 2A). FIG. 2C shows a schematic outline of a typical experiment. The medium is treated by DBD plasma for varying amounts of time (0 to about 120 seconds), depending on the desired potency of treatment. DBD plasma-treated medium is removed from the glass holder and applied to cultured cells or corneas as described in the Examples section.

FIG. 3 shows the relationship between the seconds of plasma treatment and the relative number of HSV-1 genome copies, as derived from experiments described in Example 1.

FIGS. 4A-4F show micrographs comparing various treatment options using plasma treated liquids. These data show that plasma-treated liquid stimulates, then inhibits the HSV-1 productive infection in a dose-dependent manner, as derived from experiments described in Example 1.

FIG. 5 shows the relationship between the seconds of plasma treatment of liquid and the fold change in genome copy numbers, as derived from experiments described in Example 1.

FIG. 6 shows that DBD plasma-treated medium suppresses the cytopathic effect of HSV-1 in human corneal epithelial cells. hTCEpi cells were infected at MOI 0.1 and exposed to KGM-2 medium treated with DBD plasma for 0 to 40 seconds. Control cells were neither infected nor treated. Phase-contrast images were taken at 16 hours post-infection. Photomicrophotographs are representative of at least three independent experiments.

FIG. 7 shows DBD plasma-treated medium limits the expansion of HSV-1 plaques in human corneal epithelial cell monolayers. hTCEpi monolayers were infected with KOS-GFP strain of HSV-1 at a low MOI, exposed to KGM-2 medium treated with DBD plasma for 0 to 40 seconds, and overlaid with methocellulose-containing medium to allow for plaque development. Plaques were visualized by fluorescence microscopy. A representative plaque from each treatment group is shown. Bar: 400 micron. n=2.

FIGS. 8A-B show that DBD plasma-treated medium reduces viral replication in human corneal epithelial cells. hTCEpi cells were infected at MOI 0.1 and exposed to KGM-2 medium treated with DBD plasma for 0 to 40 seconds. In FIG. 8A, total DNA was collected at 16 hours post-infection for analysis by qPCR with primers against HSV-1 polymerase and GAPDH. Bars represent relative DDC(t) values 6 SEM. In FIG. 8B. supernatants were collected at 16 hours post-infection for analysis by plaque assay. A representative experiment is shown. n=3 for all.

FIGS. 9A-B show DBD plasma-treated medium reduces accumulation of HSV-1 transcripts and protein in human corneal epithelial cells. hTCEpi cells were infected at MOI 0.1 and exposed to KGM-2 medium treated with DBD plasma for 0 to 40 seconds. Cells were collected for protein lysates or RNA isolation at 16 hours post-infection. In FIG. 9A, transcripts from all three HSV-1 gene families were detected with primers for ICP0 (immediate early), DNA polymerase (early), and glycoprotein C (true late). Bars represent relative DDC(t) values 6 SEM. In FIG. 9B, Glycoprotein C accumulation was assaved by Western blot. Nucleolin is a loading control. n=2 for all.

FIGS. 10A-10C show DBD plasma-treated medium suppresses HSV-1 replication in explanted human corneas. Human corneas were maintained in ex vivo culture as shown in the schematic (inset in FIG. 10B, expanded and shown in FIG. 10C below). Corneas were infected with 1×10⁴ PFU/cornea and exposed to KGM-2 medium treated with DBD plasma for 120 seconds. In FIG. 10A, total DNA was isolated from the epithelial layers at 24 hours post-infection and analyzed by qPCR with primers against HSV-1 DNA polymerase and GAPDH. Bars represent relative DDC(t) values 6 SEM. In FIG. 10B, culture media were collected from the same corneas and processed by plaque assay. Bars represent average viral titers 6 SEM. Data were collected from 16 matched corneas obtained from 8 donors (n=8). Procedures were as derived from Example 2.1.3.

FIGS. 11A-B show DBD plasma-treated medium exhibits low toxicity in explanted human corneas. Ex vivo human corneas were exposed to KGM-2 medium treated with DBD plasma for 120 seconds and incubated for 24 hours as derived from experiments described in Example 2.1.3. FIG. 11A shows that epithelial toxicity was assessed by fluorescein staining, with surgically de-epithelialized corneas serving as a positive staining control. n=2. In FIG. 11B, histologic assessment of toxicity was performed by examining H&E-stained tissue sections from 12 matched corneas obtained from 6 donors. Representative images from a matched pair are shown (n=6).

FIGS. 12A-B show DBD plasma-treated medium does not produce cyclobutane pyrimidine dimers or nucleic acid oxidation in explanted human corneas. Ex vivo human corneas were exposed to hydrogen peroxide (200 micro-M). UV light (20 J/m²), DBD plasma-treated KGM-2 (120 seconds), or mock treatment. The corneas were then incubated in fresh KGM-2 for 2 hours, flash-frozen in OCT compound, and processed for indirect immunofluorescence. In FIG. 12A, cyclobutane pyrimidine dimers (CPDs) were detected with the TDM-2 antibody, and In FIG. 12B, oxidized nucleic acids were detected with the 8-OHdG antibody. Nuclei were counterstained with Hoechst 33.258. Fluorescent images are overlaid with phase-contrast images in the merge. Bar: 100 micron. Representative data are shown. n=2.

FIG. 13A-B show data from two different experiments on the effect of treatment of hTCepi cells with solutions of lysates for phosphate buffered saline (Ca/Mg-free)(PBS), PBS+100 mM valine or growth media containing 10% fetal calf serum treated with micro (FIGS. 13A-13B, top panel) or nano-second discharge plasma (FIGS. 13A-13B, bottom panel) as described in Example 3. The plasma treated solutions were held for the indicated times—in FIGS. 13A for 1-60) minutes and in FIGS. 13B for 2-48 hours—prior to being added to the cells. Western blot using the indicated antibodies was performed. Gamma-H2AX is a measure of DNA damage and is used as a measure of the potency of the plasma after different holding periods.

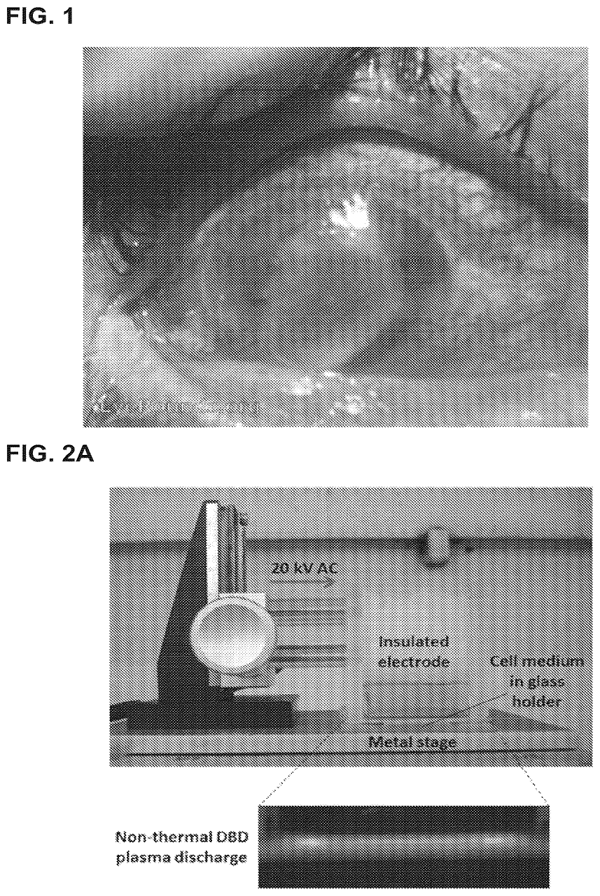

### DETAILED DESCRIPTION OF ILLUSTRATIVE EMBODIMENTS

The present invention is directed to use of plasma-treated liquids for the treatment of herpes keratitis.

The present subject matter may be understood more readily by reference to the following detailed description taken in connection with the accompanying figures and examples. which form a part of this disclosure. It is to be understood that this subject matter is not limited to the specific devices, methods, applications, conditions or parameters described and/or shown herein, and that the terminology used herein is for the purpose of describing particular embodiments by way of example only and is not intended to be limiting of the claimed subject matter.

Similarly, unless specifically otherwise stated, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer to methods of using, the compositions used in those methods, as well as the methods of manufacturing the compositions used in those methods.

When values are expressed as approximations by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, reference to values stated in ranges includes each and every value within that range.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any sub-combination. Finally, while an embodiment may be described as part of a series of steps or part of a more general composition or structure, each said step may also be considered an independent embodiment in itself.

The transitional terms “comprising.” “consisting essentially of,” and “consisting” are intended to connote their generally in accepted meanings in the patent vernacular; that is, (i) “comprising.” which is synonymous with “including.” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of” and “consisting essentially” of. For those embodiments provided in terms of “consisting essentially of,” the basic and novel characteristic(s) is the operability of the methods to treat, suppress, kill or otherwise reduce the activity herpes keratitis using plasma-treated liquids, while not peripherally injuring the eye.

Plasmas are generated by ionizing gases using any of a variety of ionization sources. Depending upon the ionization source and the extent of ionization, plasmas may be characterized as either thermal or non-thermal. Thermal and non-thermal plasmas can also be characterized by the temperature of their components. Thermal plasmas are in a state of thermal equilibrium, that is, the temperature of the free electrons, ions, and heavy neutral atoms are approximately the same. Non-thermal plasmas, or cold plasmas, are far from a state of thermal equilibrium; the temperature of the free electrons is much greater than the temperature of the ions and heavy neutral atoms within the plasma. The present application relates to the use of aqueous fluids treated with non-thermal plasmas.

Non-thermal plasmas are known to be useful for their antibacterial characteristics, because of the ions that form during the generation of the plasma, whether these ions are directly applied to tissue or where the ions are dissolved in liquids. But it has not been previously established that aqueous fluids, or any other liquid, treated with non-thermal plasma, can kill or reduce the activity of HSV-1 or HSV-2 in an eye of a patient, while not peripherally injuring that eye.

Certain embodiments of the present invention comprise methods of treating herpes keratitis, each method comprising contacting or irrigating an eye of a patient in need of such treatment with an aqueous fluid that has been previously contacted with a non-thermal plasma. In some of these embodiments, the patients are mammals, including human patients.

As used herein, the terms “treat,” “disinfect,” “disinfecting,” or the like refer to the ability or render pathogens less active, or to kill, inactivate, inhibit the growth, or otherwise render pathogens innocuous or less active, where pathogens include HSV-1 and HSV-2.

Unless otherwise specified, the term “aqueous” refers to a fluid comprising at least 95 wt % water, relative to the weight of the entire composition, the balance comprising other liquid solvents (e.g., alcohols such as ethanol or isopropanol), dissolved electrolytes or additives, or a combination thereof. However, in other independent embodiments, when specifically stated, the term “aqueous” may be used to describe a fluid comprising water in a range of from about 20 to about 30 wt %, from about 30 to about 40 wt %, from about 40 to about 50 wt %, from about 50 to about 60 wt %, from about 60) to about 70 wt %, from about 70 to about 80 wt %, from about 80 to about 90 wt %, from about 90 to about 95 wt %, from about 95 to about 100 wt %, or a combination of these ranges, in each case relative to the weight of the entire composition.

In certain embodiments, the fluid is in a liquid form. In other embodiments, the fluid is a misted or aerosolized liquid. Treatments may comprise any combination of irrigation by liquid or misted or aerosolized liquid. The irrigation may be applied statically, for example, wherein the fluid is held in a cup over the eye for prescribed time—e.g., 1 min to about 10 minutes, depending on strength of active species within the plasma-treated liquid. Alternatively or additionally, the irrigation may be applied by flowing the fluid over the eye—e.g., at a flow rate from about 0.1 mL/min to about 200 mL/min. In other embodiments, the plasma-treated fluid is absorbed in an absorbent medium (e.g., a gel or a bandage) and the medium held to contact the fluid with the eye.

Suitable aqueous liquids include saline, deionized water, tap water, and phosphate buffered saline (PBS), and growth media (e.g., KGM-2 growth medium) among others. The irrigating fluid may comprise salts or additives which assist in the treatment of the herpes keratitis or other associated or coincident afflictions. For example, in some embodiments, the fluid comprises saline, buffering agents (e.g., phosphate buffer), growth media (e.g., KGM-2 growth medium), anti-oxidants, or a combination thereof. The fluid may also contain local anesthetics, colorants, or other antimicrobial agents to support the patient treatment, provided these additives do not significantly compromise the activity of the plasma-treated fluid for its intended purpose of treating the herpes keratitis.

The efficacy of the plasma-treated fluid depends on the type and intensity of the plasma, the nature of the fluid, and the duration of plasma treatment.

In certain embodiments, the non-thermal plasma is derived from a dielectric barrier discharge, a corona or pulsed corona discharge, arc, spark, gliding arc, radio frequency discharge, microwave discharge or any combination thereof. Each of these plasma types are well known in the art. Dielectric barrier discharge plasmas are preferred.

In certain embodiments, the non-thermal plasma, is a dielectric barrier discharge (DBD). A DBD may be generated using an alternating current at a frequency of from about 0.5 kHz to about 500 kHz between a high voltage electrode and a ground electrode. In certain embodiments, the frequency is in a range having a lower boundary value of about 0.3 kHz, about 0.5 kHz, about 1 kHz, about 2.5 kHz, about 5 kHz, or about 10 KHz and having an upper boundary value of about 500 kHz, about 250 KHz, about 100 kHz, about 50 kHz, about 25 KHz, or about 10 KHz. Other non-limiting exemplary embodiments include the ranges 0.3 kHz to about 10 kHz or about 0.5 kHz to about 5 kHz or about 0.5 Hz to about 2 kHz. It should be noted that in certain configurations, a single pulse may be used. Therefore, the present subject matter may be preferably used in applications ranging from a single pulse to about 500 KHz. In addition, one or more dielectric barriers are placed between the electrodes. Exemplary surface power density outputs may be from about 0.001 Watt/cm² to about 100 Watt/cm². In some embodiments, the surface power density outputs may be from about 0.001 Watt/cm² to about 0.01 Watt/cm², from about 0.01 Watt/cm² to about 0.1 Watt/cm², from about 0.1 Watt/cm² to about 1 Watt/cm^{2,} from about 1 Watt/cm² to about 10 Watt/cm^{2,} from about 10 Watt/cm² to about 100 Watt/cm^{2,} or any combination thereof.

Various materials can be utilized for the dielectric barrier. These include plastic, glass, quartz, and ceramics, among others. The clearance between the discharge gaps is typically between about 0.01 mm and 5 mm (or to several centimeters). The required voltage applied to the high voltage electrode varies depending upon the pressure and the clearance between the discharge gaps. For a DBD at atmospheric pressure and a few millimeters between the gaps, the voltage required to generate a plasma may vary, but in some configurations, is about 10 kV. In some embodiments, the voltage used to generate the non-thermal plasma is in a range of from about 1 kV to about 5 kV, from about 5 kV to about 10 kV, from about 10 kV to about 15 kV, from about 15 kV to about 20 kV, from about 20 kV to about 25 kV, from about 25 kV to about 30 kV, from about 35 kV to about 40 kV, from about 40 kV to about 50 kV, or a combination thereof.

In certain embodiments, the plasma may be generated having a surface energy (i.e., at the surface of a plate electrode) of at least about 0.1 J/cm². In other embodiments, the plasma may have a surface energy of at least about 0.5 J/cm^{2,} at least about 1 J/cm^{2,} at least about 5 J/cm^{2,} at least about 10 J/cm^{2,} or at least about 20 J/cm² to about 25 J/cm^{2.} In other embodiments, the fluid that is contacted with the non-thermal plasma for a time in a range of from about 5 seconds to about 1 minute, from about 1 minute to about 5 minutes, from about 5 minutes to about 10 minutes, from about 10 minutes to about 15 minutes, or any combination thereof so as to generate the plasma-treated fluid. The energy of the plasma and the duration of its application will vary depending upon the initial strength required, the additives within the fluid, and the anticipated shelf-life of the fluid. The skilled artisan would be well positioned to determine the specific energy to be used and the duration of the application.

In certain specific embodiments, the fluid is treated with a non-thermal plasma for 1 to 5 minutes. In some embodiments, the fluid is treated with a non-thermal plasma using a configuration providing a maximum frequency in a range of from about 0.5 to about 2 KHz, preferably about 1 kHz. In other embodiments, the fluid is treated with a non-thermal plasma using a configuration providing an amplitude in a range of from about 5 to 25 kV, preferably about 12.5 to 17.5 kV, or about 15 to about 20 kV. In a more specific embodiment, the fluid is treated with non-thermal plasma for 1 to 5 minutes, using a configuration providing a maximum frequency in a range of from about 0.5 to about 2 kHz, preferably about 1 kHz, at an amplitude in a range of from about 5 to 25 kV, preferably about 12.5 to 17.5 kV or about 15 to 20 kV.

One benefit of the present subject matter is the ability to apply the plasma-treated liquid remote from a plasma source. For example, the treating fluid may be contacted with the plasma to form a disinfecting composition and the disinfecting composition may be subsequently transported to another location for contacting with the patient's eye(s). See. e.g., Example 3. For example, the disinfection composition may be formed and transported to a different location within a laboratory or other room, or it may be transported to an entirely different building. Thus, in certain embodiments, the eyes may be irrigated with the disinfection composition for a period of time after the disinfection composition is formed. In certain embodiments, this period of time may be in a range of from about 1 to about 5 minutes, about 5 minutes to about 10 minutes, from about 10 minutes to about 20 minutes, from about 20 minutes to about 30 minutes, from about 30 minutes to about 60 minutes, from about 60 minutes to about 90 minutes, from about 90 minutes to about 120 minutes, or any combination thereof, or even longer.

Depending on the strength of the as-used plasma-activated materials in the fluid and the activity of the HSV-1 or HSV-2 viruses, the length of the time of irrigation or contact may vary. In certain embodiments, this period of time may be in a range of from about 10 seconds to about 1 minute, from about 1 minute to about 5 minutes, about 5 minutes to about 10 minutes, from about 10 minutes to about 20 minutes, from about 20 minutes to about 30 minutes, from about 30 minutes to about 60 minutes, from about 60 minutes to about 90 minutes, from about 90 minutes to about 120 minutes, or any combination thereof, or even longer. Once the eye(s) is/are contacted with a plasma-treated liquid, the disinfection material may remain in contact with the surface for a period of time that may be referred to as a “treatment time.” In certain embodiments, the treatment time may be at least about 5 seconds, or at least about 30 seconds, or at least about 60 seconds, or at least about 600 seconds until the time is no longer efficacious.

Also, given the persistence and ubiquity of the HSV-1 and HSV-2 viruses, the methods may be applied over a regular course of treatments. Specific embodiments provide that the irrigation, using at least one of the embodiments already described, is done at least 2, 3, 4, 5, 10, or 20 times, depending on the efficacy of the treatment. It would be well within the skill of the skilled practitioner to determine the necessary course of treatment, without undue experimentation.

The extent of disinfection depends upon factors such as the type and amount of plasma-treated material, plasma energy, and exposure time, among others. In certain embodiments, the treating is sufficient to reduce the number of HSV-1 or HSV-2 genome copies in the eye, relative to the number of HSV-1 or HSV-2 genome copies before treatment. In other embodiments, the treating reduces the number of HSV-1 genome copies in the eye by at least 20%, by at least 40%, by at least 60%, or by at least 80%, relative to the number of HSV-1 genome copies before treatment.

In some embodiments, the methods described herein may be used to treat or disinfect HSV-1 or HSV-2 viruses on other parts of the body, such as mucosal surfaces (including lips, mouth and genitalia), or on inanimate surfaces.

The following embodiments are intended to complement, rather than supplant, those embodiments already described.

### Embodiment 1

A method of treating herpes keratitis comprising irrigating an eye of a patient in need of such treatment with an aqueous fluid that has been previously contacted with a non-thermal plasma.

### Embodiment 2

The method of Embodiment 1, wherein the treating reduces the number of HSV-1 genome copies in the eye, relative to the number of HSV-1 genome copies before or without treatment.

### Embodiment 3

The method of Embodiment 1 or 2, wherein the treating reduces the number of HSV-1 genome copies in the eye by at least 20%, by at least 40%, by at least 60%, or by at least 80%, relative to the number of HSV-1 genome copies before treatment.

### Embodiment 4

The method of any one of Embodiments 1 to 3, wherein the fluid is a liquid.

### Embodiment 5

The method of any one of Embodiments 1 to 4, wherein the fluid is a misted or aerosolized liquid.

### Embodiment 6

The method of any one of Embodiments 1 to 5, wherein the aqueous fluid comprises saline, phosphate buffer, or a combination thereof.

Embodiment 7

The method of any one of Embodiments 1 to 6, wherein the non-thermal plasma is derived from a dielectric barrier discharge, a corona or pulsed corona discharge, arc, spark, gliding arc, radio frequency discharge, microwave discharge or any combination thereof.

### Embodiment 8

The method of any one of Embodiments 1 to 7, wherein the plasma is a non-thermal plasma having an intensity of at least about 0.1 J/cm² at the surface of a plasma source electrode.

### Embodiment 9

The method of any one of Embodiments 1 to 8, wherein the fluid that has been contacted with the non-thermal plasma for a time in a range of from about 5 seconds or 40 seconds to about 5 minutes.

### Embodiment 10

The method of any one of Embodiments 1 to 9, wherein the irrigating is done within a time in a range of from about one minute to about 10 minutes after the fluid has been contacted with the non-thermal plasma.

### Embodiment 11

The method of any one of Embodiments 1 to 10, wherein the irrigating is done for a period in a range of from about 5 seconds to about 5 minutes.

### Embodiment 12

The method of any one of Embodiments 1 to 11, wherein the irrigating is done two or more times.

### Embodiment 13

The method of any one of Embodiments 1 to 12, wherein the non-thermal plasma is derived from a dielectric barrier discharge.

### Embodiment 14

The method of any one of Embodiments 1 to 13, wherein the non-thermal plasma is generated using a configuration providing a maximum frequency in a range of from about 0.5 to about 2 kHz, preferably about 1 kHz.

### Embodiment 15

The method of any one of Embodiments 1 to 14, wherein the non-thermal plasma is generated using a configuration providing an amplitude in a range of from about 5 to 25 KV, preferably about 12.5 to 17.5 kV or about 15 to about 20 kV.

The present subject matter is further defined in the following Examples. It should be understood that these examples, while indicating specific embodiments of the subject matter, are not intending to limit the scope of the invention. From the above discussion and these examples, one skilled in the art can ascertain the essential characteristics of this subject matter, and without departing from the spirit and scope thereof, can make various changes and modifications of the subject matter to adapt it to various usages and conditions. Such modifications are considered to be within the scope of the present invention.

### EXAMPLE

### Example 1

In one non-limiting example, cultured human corneal cells were infected with HSV-1 KOS at an MOI of 0.1 for 1 hour, then washed twice with 1×PBS. One milliliter of KGM-2 growth medium was treated with non-thermal plasma for between 5 and 60 seconds at maximum frequency (1000 Hz) and amplitude (15.5 kV). Cells were exposed to 0.8 mL of plasma-treated medium for 5 minutes, after which 5 mL of untreated medium was added. Cells were photographed and subsequently isolated at various time points for assay of viral copy number, viral titre by plaque formation assays and gene expression.

In related experiments, the times of treating liquids with plasma were varied from 0 to 120 seconds; the post infection sampling times were varied.

To measure viral titers, growth media was collected 24 hpi, sterile filtered, and standard plaque assay was performed.

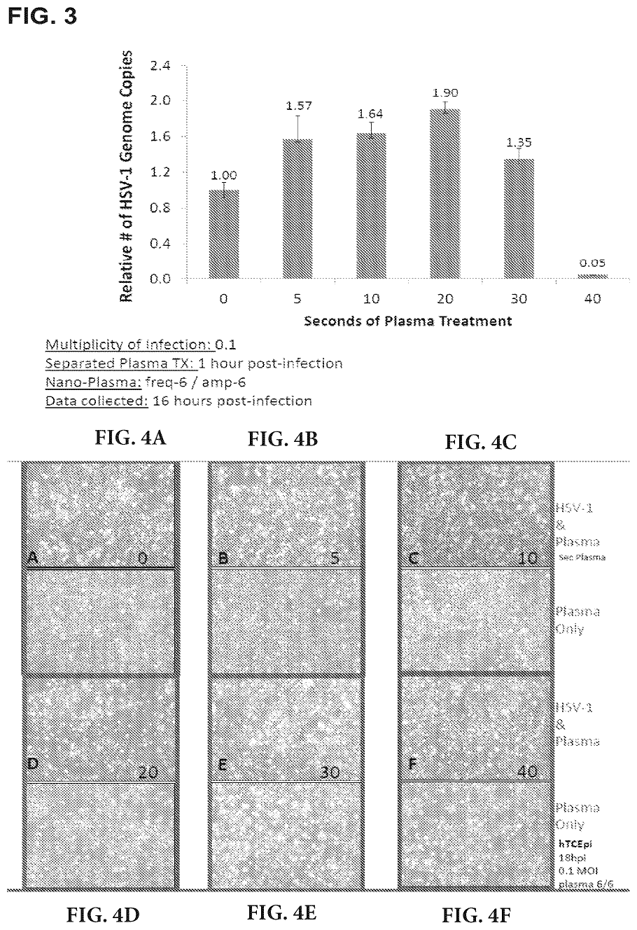

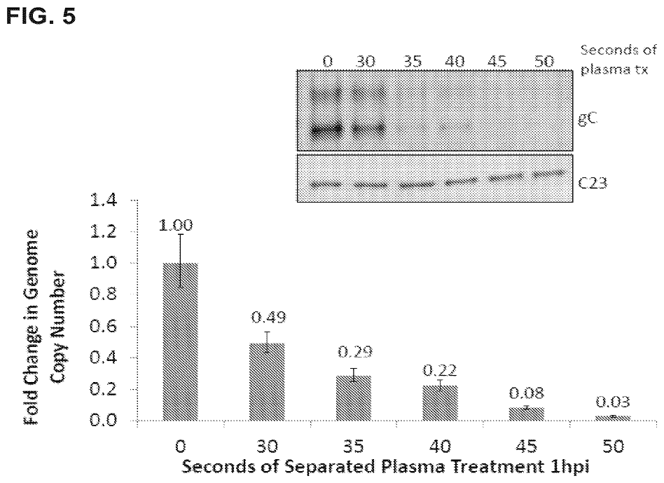

The resulting data are shown in FIG. 3 through FIG. 5.

### Example 2

### Example 2.1: Methods

### Example 2.1.1: Cells and Viruses

All cells were cultured at 37° C. and 5% CO₂, and supplemented with 100 U/mL penicillin and 100 micro-g/mL streptomycin. Human corneal epithelial cells immortalized with hTERT (hTCEpi; as described in Robertson D M, et al. Characterization of growth and differentiation in a telomerase-immortalized human corneal epithelial cell line. Invest Ophthalmol Vis Sci. 2005;46: 470-478; a kind gift from James Jester at University of California-Irvine) were grown in complete keratinocyte growth medium 2 (KGM-2; Lonza, Basel, Switzerland). African green monkey kidney fibroblasts (CV-1; as described in Jensen F C, et al., Infection of human and simian tissue cultures with Rous sarcoma virus. Proc. Natl Acad Sci U S A. 1964;52:53-59; American Type Culture Collection, Manassas, VA) were grown in Dulbecco's modified Eagle's medium (DMEM; Cellgro, Manassas, VA) supplemented with 10% fetal bovine serum (FBS). KOS strain of HSV-1 (as described in Smith K O. Proc Soc Exp Biol Med. 1964;115:814-816; a kind gift from Stephen Jennings at Drexel University College of Medicine) was used throughout, except for the plaque expansion experiment, which was performed with KOS-GFP strain (as described in Lock M, Miller C, Fraser N W, J. Virol. 2001;75: 3413-3426; a kind gift from Nigel Fraser at University of Pennsylvania). All viral stocks were titered on CV-1 monolayers.

### Example 2.1.2: Cell Culture Model

Subconfluent monolayers of hTCEpi cells were grown in six-well plates. Infections with KOS strain of HSV-1 were carried out at multiplicity of infection (MOI) 0.1 in a 200-micro-L inoculum volume at 37° C. for 1 hour with intermittent rocking. The infected monolayers were then exposed to DBD plasma-treated medium (as described below) and overlaid with fresh KGM-2 for the remainder of experiment. At 16 hours post-infection, phase-contrast images were taken; cells were collected for isolation of DNA, RNA, or protein; and culture medium was collected for plaque assays.

For plaque expansion experiments, hTCEpi monolayers were infected with KOS-GFP strain of HSV-1, which constitutively expresses green fluorescent protein (GFP) from a cytomegalovirus immediate early promoter. Infections were carried out at very low MOI to ensure that the viral plaques would be sufficiently sparse. Following exposure to DBD plasma-treated medium, cells were overlaid with fresh KGM-2 containing 1.25% wt/vol methocellulose. Infectious plaques were then allowed to develop and were imaged by fluorescence microscopy.

### Example 2.1.3: Corneal Explant Model

Human corneas were obtained from the Lions Eye Bank of Delaware Valley. Experimentation using human corneas was approved by the Drexel University College of Medicine Institutional Review Board and adhered to the tenets of the Declaration of Helsinki. Protocol established by Alekseev et al. J. Vis. Exp., 2012; e3631 for ex vivo corneal culture, infection, and treatment was followed closely. Briefly, corneoscleral buttons were rinsed in PBS containing 200 U/mL penicillin and 200 micro-g/mL streptomycin. The endothelial concavity was filled with culture medium containing 1% low melting temperature agarose. The corneas were cultured epithelial side up in KGM-2 medium supplemented with 200 U/mL penicillin and 200 micro-g/mL streptomycin. The next day, they were infected (corneal side down) with 1×10⁴ plaque forming units (PFU)/cornea of strain KOS HSV-1 for 1 hour, exposed to DBD plasma-treated medium (as described). At 2 hours post-infection (hpi), corneas were washed twice with 1×PBS, rotated epithelial side up, and placed in normal growth medium overnight. At 24 hpi, the corneal epithelial surface was scraped in 200 μl PBS. DNA was isolated and analyzed for HSV-1 genome copy number by qPCR; GAPDH was used as a reference gene. Raw data were analyzed by the ΔΔCt method.

### Example 2.1.4: Generation of DBD Plasma in Atmospheric Air

To initiate uniform DBD in atmospheric air, a nanosecond-pulsed power system was used. The power supply (FID GmbH, Burbach, Germany) generated pulses with ±15.5-kV pulse amplitude in a 50 ohm coaxial cable (31 kV on the high-voltage electrode due to pulse reflection), 10-ns pulse duration (90% amplitude), 2-nanosecond rise time, and 3-nanosecond fall time. Power measurements were performed using a high-voltage probe (P6015A, 75-MHz bandwidth; Tektronix, Beaverton, OR) and Pearson current monitor (model 6585, usable time 1 nanosecond, 1 GHz bandwidth; Palo Alto, CA) connected to a 1-GHz oscilloscope (DPO4104B; Tektronix). Because the probe's frequency response limited its ability to accurately detect the pulse shape, voltage was measured using back current shunts (BCS). For this purpose, pulses were delivered to the electrodes via 30 m of RG 393/U high-voltage coaxial cable, and BCS was mounted 6.7 m from the output of the power supply. The shunt comprised 10 carbon-composition 3 ohm resistors (OF30GJE-ND; DigiKey, Thief River Falls, MN) soldered into a gap within the shield of the cable. Amplitude calibration of the BSC was performed using a high-voltage probe. In order to account for the displacement current, which was later subtracted from the total current of the discharge, measurements were first done with a large electrode gap when the electric field in the gap was not sufficient to generate a discharge and therefore only displacement current could be measured. These measurements were also confirmed by a well-established technique for estimation of energy deposition based on comparison of the first incident and reflected voltage pulses in a long cable. These measurements estimate the resulting DBD pulse energy at 45±4 mJ. Liquid treatment experiments were performed at a frequency of 1 kHz, corresponding to the total plasma discharge power of 1.88±0.2 W/cm².

Dielectric barrier discharge optical emission spectrum was obtained using a fiber optic bundle (10 fibers, 200-micron core) connected to a spectrometer system (TriVista TR555) with a digital intensified charge-coupled device (ICCD) camera (PI-MAX), all purchased from Princeton Instruments (Trenton, NJ). The rotational temperature of nitrogen, which represents the gas temperature was determined by fitting a synthetic spectrum to the experimental spectrum of the (0-2) transition emission bands of the N₂ (C³ Πu-B ³Πg) transition (second positive system) in the range 360 to 381 nm, using the Specair 3.0 program (SpectralFit S.A.S., Antony, France). The measured rotational temperature of nitrogen was 343±6 K.

### Example 2.1.5: Non-thermal DBD Plasma Treatment of Liquids

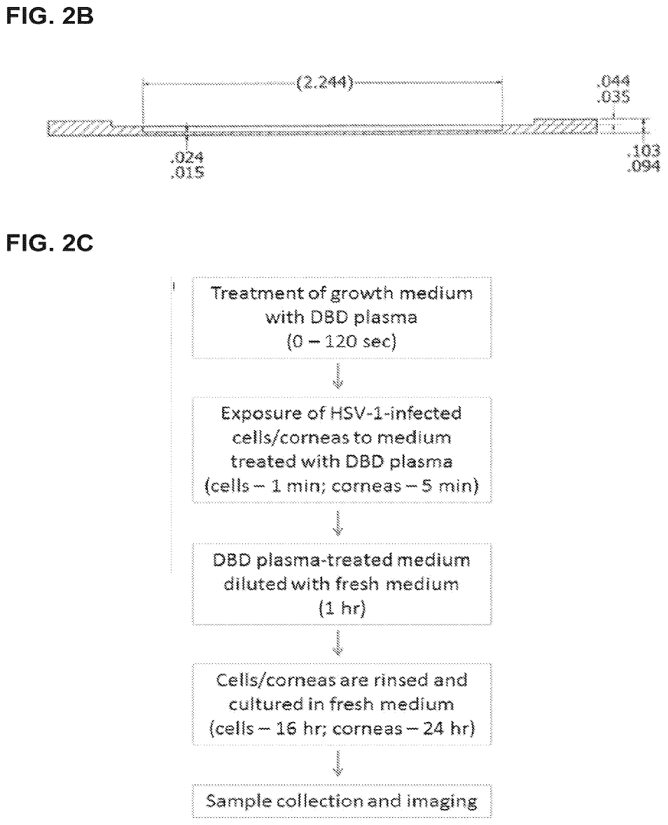

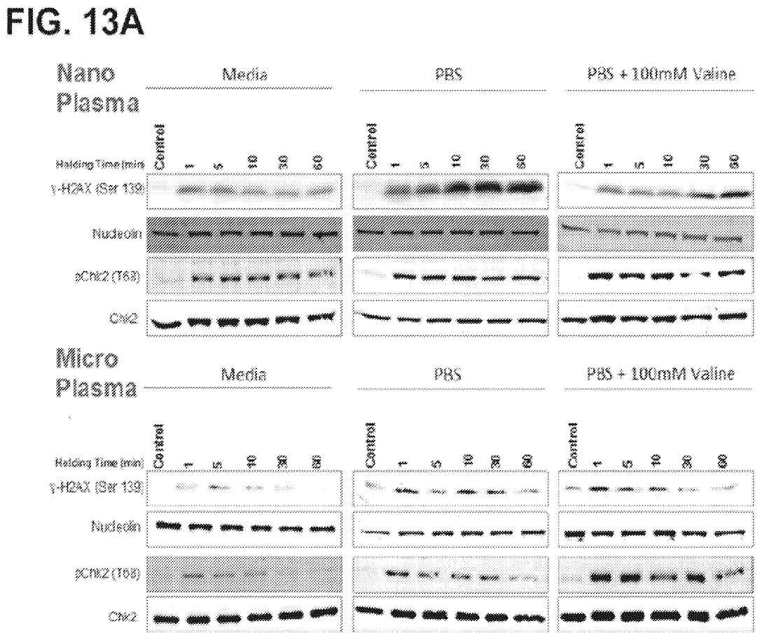

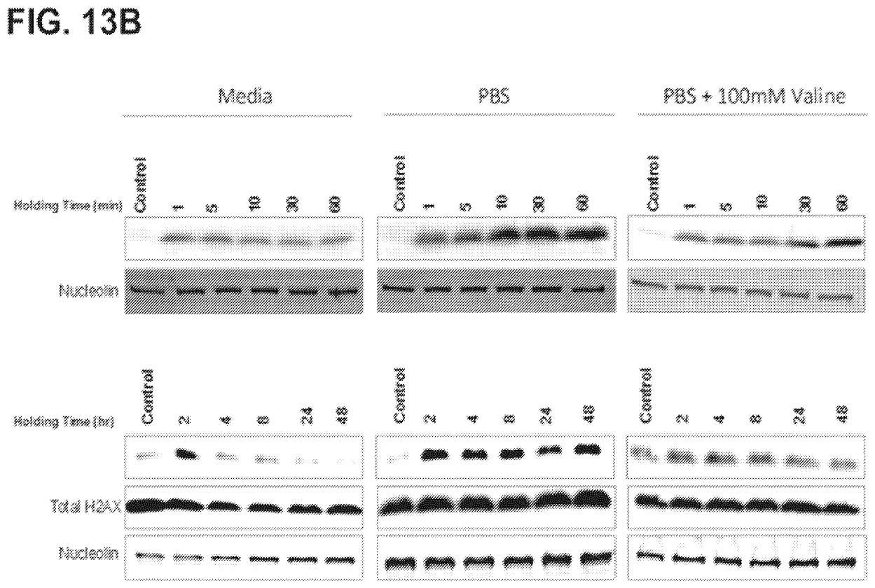

Dielectric barrier discharge plasma-treated liquid was generated by exposing 1 mL complete KGM-2 medium in a glass holder (FIG. 2B) to DBD plasma, as shown in FIG. 2A. Additional experiments shown in FIGS. 13A/B involved the treatment of Ca/Mg-free phosphate buffered saline (PBS) or PBS+100 mM valine. The potency of plasma-treated liquid was adjusted by varying the duration of treatment time (0-180 seconds). Once treated, the liquid was applied to cells or corneas (400 micro-L for cells, 800 micro-L for corneas) in six-well plates at exactly 1 hour after the start of HSV-1 infection. Cells were incubated with the DBD plasma-treated medium for 1 minute and corneas for 5 minutes before fresh KGM-2 (2 mL for cells, 6 mL for corneas) was added to each well to dilute the DBD plasma-treated medium. After 1 hour, cells or corneas were rinsed and overlaid with fresh KGM-2 for the remainder of the experiment (FIG. 2C). Similarly, treatment of PBS or PBS+ valine followed the same protocol (FIG. 13).

### Example 2.1.6: Corneal Toxicity Assessment

Explanted human corneas not infected with HSV-1 were exposed to DBD plasma-treated medium in the same manner as described above and were subsequently cultured for 24 hours. For histology studies, corneas were fixed in 3% paraformaldehyde/2% sucrose solution, paraffin embedded, sectioned, and stained with hematoxylin and eosin (H&E). For assessment of epithelial toxicity, corneas were briefly stained with fluorescein (1% wt/vol in PBS), and epithelial defects were imaged with 464-nm-wavelength blue light (LDP LLC, Carlstadt, NJ).

### Example 2.1.7: Genotoxic Toxicity Assessment

Explanted human corneas were exposed to hydrogen peroxide (200 micro-M), UV light (20 J/m²), DBD plasma-treated KGM-2 (120 seconds), or mock treatment. The corneas were then incubated in fresh KGM-2 for 2 hours and flash-frozen in optimal cutting temperature (OCT) compound. Frozen tissue blocks were sectioned at 5-1 m thickness, fixed, and processed for indirect immunofluorescence. Detection of cyclobutane pyrimidine dimers with the TDM-2 primary antibody (a kind gift from Toshio Mori at Nara Medical University, Japan) was performed according to a previously published protocol of Kalghatgi S, et al., “Effects of non-thermal plasma on mammalian cells.” PloS ONE. 2011;6:e16270. Oxidative damage to nucleic acids was assessed by staining with the 8-OHdG primary antibody (Santa Cruz Biotechnology, Santa Cruz, CA), which detects 8-hydroxy-20-deoxyguanosine, 8-hydroxyguanine, and 8-hydroxyguanosine. Standard immunofluorescence protocol was followed. Nuclei were counterstained with Hoechst 33,258.

### Example 2.1.8: Viral Genome Replication and Transcription

Viral genome replication and transcription were measured by qPCR. Total DNA and RNA from infected cells were isolated using the DNeasy Blood & Tissue Kit and the RNeasy Mini Kit, respectively (Qiagen, Hilden, Germany). RNA was converted to cDNA using qScript (Quanta BioSciences, Gaithersburg, MD). Real-time qPCR was performed with SYBR Green (Bio-Rad, Hercules, CA). Target primers for UL30 (DNA polymerase catalytic subunit) and reference primers for glyceraldehyde 3-phosphate dehydrogenase (GAPDH) were used to measure genome replication. Transcription of the three gene families was measured with primers for RL2 (ICP0), UL30 (DNA polymerase catalytic subunit), and UL44 (gC), with reference primers for the 18S rRNA (Table 1). All primer sequences have been previously published. (SEQ ID NOS 1-10, respectively, in order of appearance).

TABLE 1
PCR Primers Used in this Study

| | Primer | SEQ. | |
|---|---|---|---|
| Target | Direction | ID. No | Primer sequence (5′→3′) |
| ICP0 | Fwd | 1 | CTG CGC TGC GAC ACC TT |
| | Rev | 2 | CAA TTG CAT CCA GGT TTT CAT G |
| DNA | Fwd | 3 | AGA GGG ACA TCC AGG ACT TTG T |
| polymerase | Rev | 4 | CAG GCG CTT GTT GGT GTA C |
| Glycoprotein | Fwd | 5 | ATT CCA CCC GCA TGG AGT TC |
| C | Rev | 6 | CGG TGA TGT TCG TCA GGA CC |
| GAPDH | Fwd | 7 | GCT TGC CCT GTC CAG TTA AT |
| | Rev | 8 | TAG CTC AGC TGC ACC CTT TA |
| 18S rRNA | Fwd | 9 | GTA ACC CGT TGA ACC CCA TT |
| | Rev | 10 | CCA TCC AAT CGG TAG TAG CG |

### Example 2.1.9: Western Blot

Standard protocol was followed for Western blot analysis. Cell lysates were collected in 200 micro-L Laemmli buffer, vortexed, and boiled at 95° C. for 5 minutes. Protein concentrations were measured by bicinchoninic acid assay. SDS-PAGE was followed by transfer onto a polyvinylidene fluoride membrane, which was then blocked in 5% BSA. Blots were stained with primary antibodies against glycoprotein C (rabbit polyclonal; a kind gift from Roselyn Eisenberg at University of Pennsylvania) and nucleolin (mouse monoclonal; Santa Cruz Biotechnology). Blots were stained with secondary antibodies and visualized with the Odyssey near-infrared system (LICOR, Lincoln, NE).

### Example 2.1.10: Statistical Analysis

Statistical significance was determined using Student's t-test and is indicated by ns (P>0.05), *(P<0.05), **(P<0.01), or ***(P<0.001).

### Example 2.2: Results

### Example 2.2.1: DBD Plasma-Treated Medium Suppresses HSV-1 Infection in Human Corneal Epithelial Cells

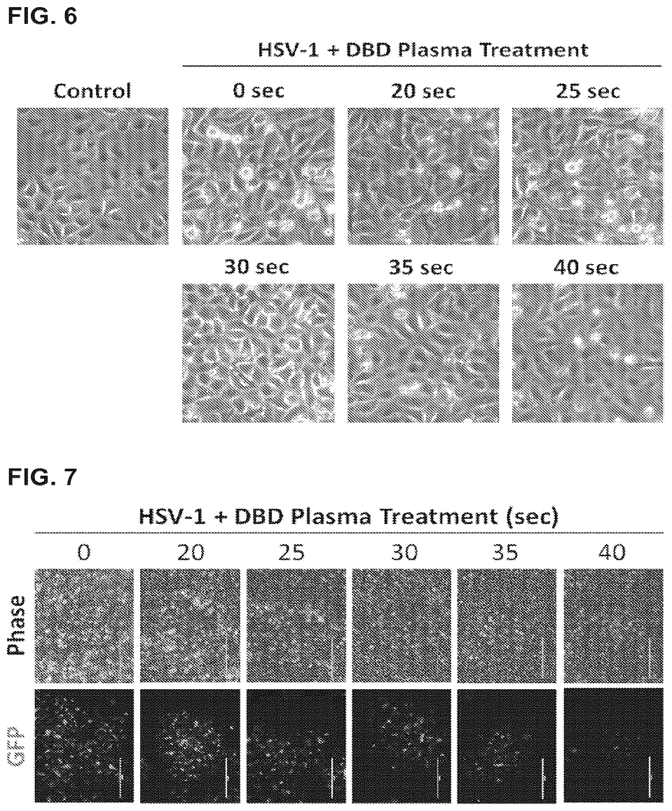

In order to obtain a general characterization of the effect of DBD plasma on HSV-1 infection, experiments were conducted using hTCEpi corneal epithelial cells. Monolayers of hTCEpi cells were infected with HSV-1 at low MOI (0.1) to simulate physiologically relevant viral titers. KGM-2 growth medium was treated with DBD plasma for 0 to 40 seconds and then applied to the infected cells as described in Methods (FIGS. 2A-C). This range of treatment times was chosen based on our previous studies of biological effects of DBD plasma (data not shown). The cytopathic effect produced by HSV-1 infection was suppressed by DBD plasma-treated medium in a dose-dependent manner, with the maximal antiviral activity achieved at 35 to 40 seconds of DBD plasma treatment (FIG. 6). To gain better understanding of this antiviral effect, the spread of HSV-1 infection was monitored within the hTCEpi monolayers. To this end, confluent hTCEpi cells were infected with KOS-GFP strain of virus, which constitutively expresses GFP allowing for easy visual detection of infected cells. The monolayers were overlaid with methocellulose-containing medium, limiting viral infection to direct spread. Examination of the infectious plaques by fluorescence microscopy revealed that DBD plasma greatly limited HSV-1 plaque expansion (FIG. 7).

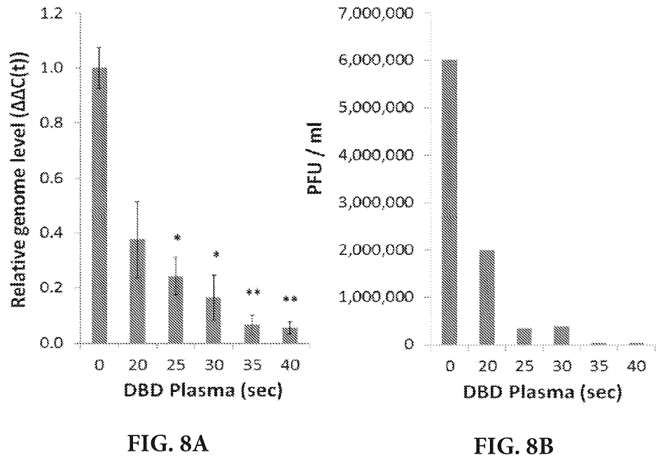

To provide a quantitative evaluation of the antiviral effect of DBD plasma, a qPCR was used assay for the measurement of viral genome replication, hTCEpi monolayers that had been exposed to DBD plasma-treated medium contained significantly lower HSV-1 genome copies than control monolayers. The inhibition of genome replication was greater than 90% at the 40-second treatment intensity (FIG. 8A). Culture media from the same monolayers were analyzed by plaque assay, revealing a concomitant inhibition of infectious viral particle production, which reached 150-fold reduction at the 40-second treatment intensity (FIG. 8B). Consistent with the initial assessment of the cytopathic effect (FIG. 6), the maximal effect on both the genome replication and the viral titers was achieved at the 35- to 40-second treatment intensity.

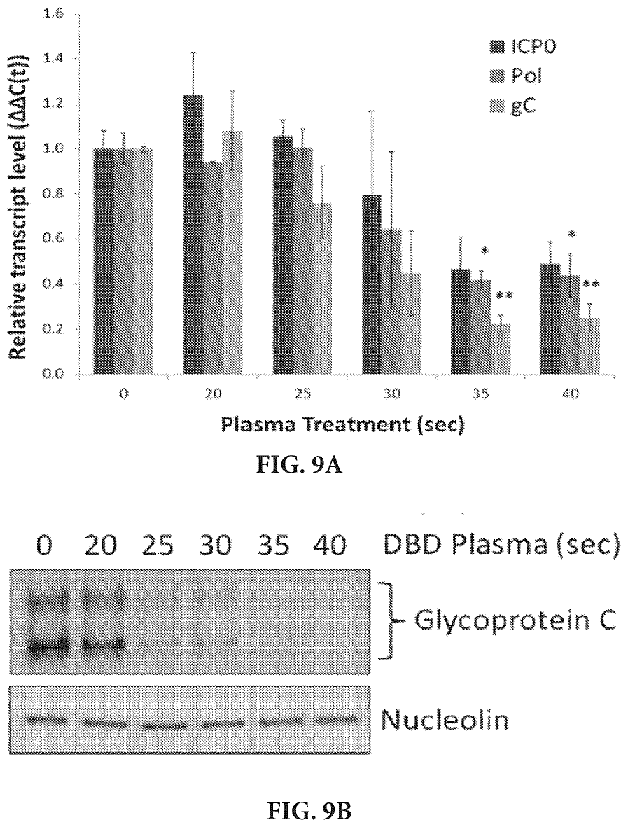

The inhibition of genome replication caused a subsequent reduction in the accumulation of viral gene products. Levels of viral transcripts from all three kinetic families—mmediate early, early, and late-were reduced, as measured by qRT-PCR with primers against RL2 (ICP0), UL30 (DNA polymerase catalytic subunit), and UL44 (glycoprotein C) (FIG. 9A). There was a consistent reduction in the accumulation of glycoprotein C protein product as detected by Western blot (FIG. 9B). Interestingly, the decrease of glycoprotein C protein levels was more pronounced than the decrease of its mRNA transcript, which could point to an unexplored translational effect of DBD plasma.

Taken together, the experiments in the corneal tissue culture model revealed a potent antiviral effect of DBD plasma. The 35- to 40-second treatment intensity resulted in pronounced reduction of the cytopathic effect, infectious plaque expansion, viral genome replication, production of infectious progeny, and accumulation of viral gene products.

### Example 2.2.2: DBD Plasma-Treated Medium Suppresses HSV-1 Infection in Explanted Human Corneas

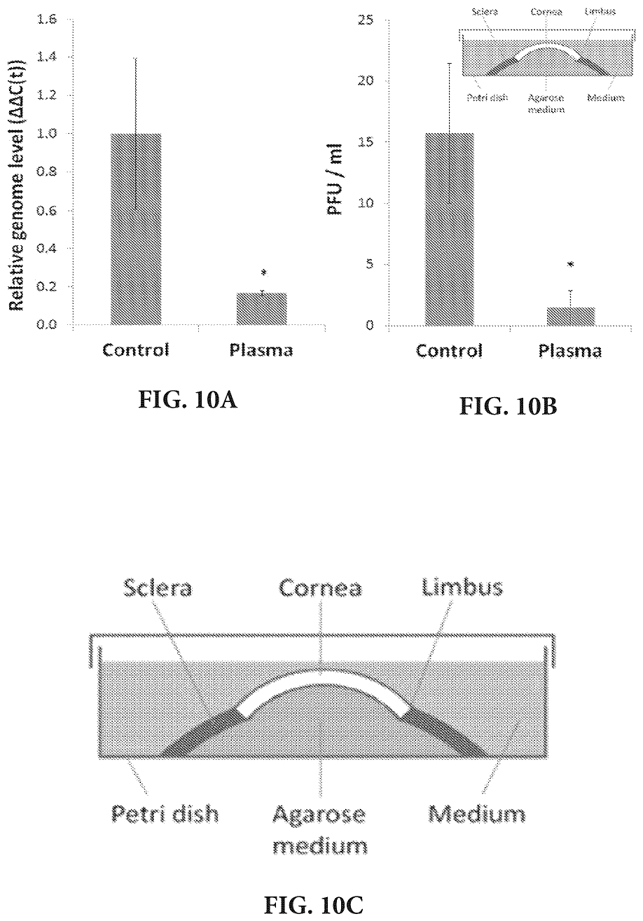

In order to extend these experiments to a more physiologically relevant model of corneal HSV-1 infection, the method of Alekseev et al. J. Vis. Exp., 2012;e3631 was used for ex vivo corneal culture, infection, and treatment (inset in FIG. 10B). Intact human corneoscleral buttons were infected with HSV-1 and exposed to DBD plasma-treated medium similarly to the in vitro experiments. Due to the inherent differences between cell monolayers and explanted corneas, a new dose-response curve was generated (data not shown); based on the ex vivo dose response, 120 seconds was chosen as the optimal treatment intensity to be used in subsequent experiments. A set of 16 donor-matched human corneas was infected and exposed to medium treated with DBD plasma or mock (DBD plasma power source turned off). A substantial (over 80%) reduction in HSV-1 genome replication was achieved in treated corneas compared to matched mock-treated controls (FIG. 10A). This decrease was accompanied by a similar reduction of the viral load in the culture medium (FIG. 10B). Thus, the initial in vitro findings (FIGS. 3-5) were supported by the ex vivo experiments in intact human corneas.

### Example 2.2.3: DBD Plasma-Treated Medium Exhibits Low Toxicity in Explanted Human Corneas

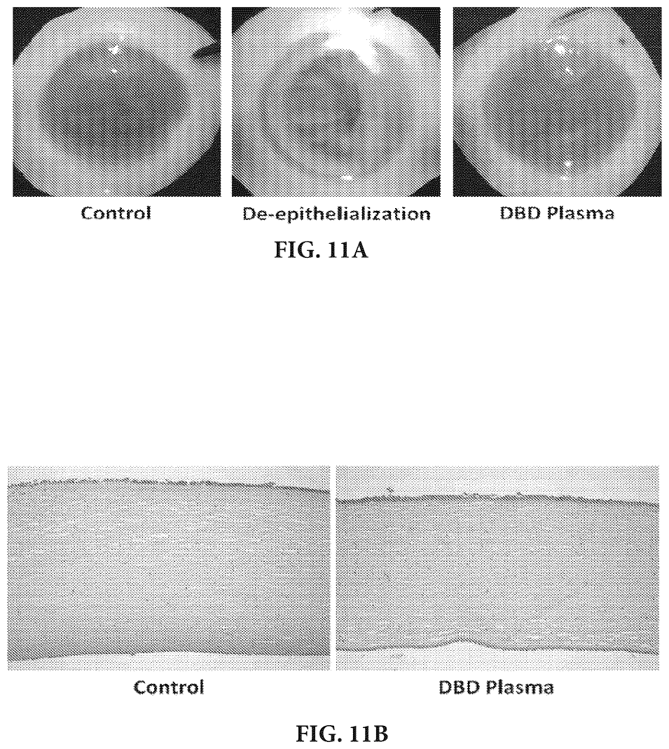

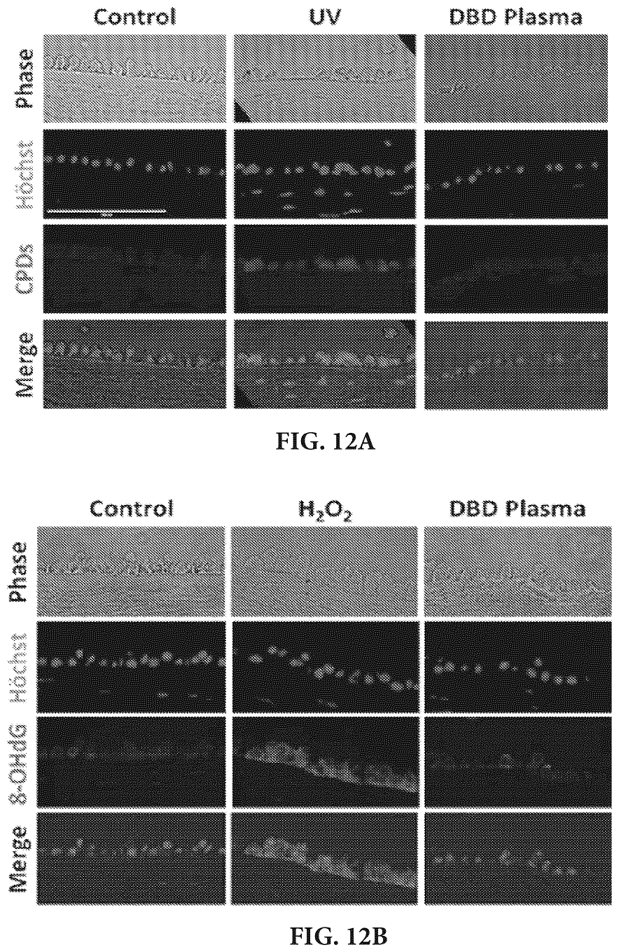

Brun et al., PloS ONE. 2012;7:e33245 have performed comprehensive and extensive toxicity studies, demonstrating a lack of pronounced or lasting detrimental effects of non-thermal plasma to the human cornea. However, since the method of plasma treatment utilized in the present study is different from the helium-flow plasma used by Brun et al., additional toxicity assessment was necessary. Explanted human corneas were exposed to DBD plasma-treated medium (120) seconds) and subsequently cultured under conditions identical to those in virus-inhibition experiments (FIGS. 10A-10C). At 24 hours post-treatment, the integrity of corneal epithelium was assessed by fluorescein staining, which revealed no observable abnormalities (FIG. 11A). In addition, a set of 12 donor-matched corneas was exposed to mock-treated or DBD plasma-treated medium and examined for histologic changes in the corneal structure. In agreement with the fluorescein staining, no consistent abnormalities were visually detectible in the H&E-stained tissue sections (FIG. 11B). Plasmas have been shown to produce reactive oxygen and nitrogen species, as well as a minor amount of UV energy. These entities are known to be damaging to cells and can be particularly deleterious to the nucleic acids (especially DNA) by catalyzing mutagenic structural changes. In particular, UV exposure promotes the formation of aberrant structures known as cyclobutane pyrimidine dimers (CPDs), and oxidation of nucleic acids can promote inappropriate nucleotide substitutions in the genome. For this reason, we examined the potential toxic effects of DBD plasma-treated medium on the nucleic acids of corneal epithelial cells. Explanted human corneas were exposed to DBD plasma-treated medium, mock-treated medium, or damaging agents for positive control (UV and H₂O₂). Not surprisingly, DBD plasma-treated medium did not induce the formation of CPDs, as detected by staining with an antibody specific to these structures (FIG. 12A). Importantly, the same DBD plasma treatment intensity that produces viral inhibition (120 seconds) did not induce appreciable levels of nucleic acid oxidation within the epithelium, as detected by staining with an antibody specific to 8-OHdG, a common marker of oxidative damage (FIG. 12B). Taken together, these experiments demonstrate that DBD plasma-treated medium suppresses HSV-1 infection in explanted human corneas without producing appreciable toxicity, as monitored by fluorescein staining, histologic assessment, and detection of genotoxic damage.

### Example 2.3: Discussion

The use of non-thermal plasmas in biomedical applications holds significant promise and has generated much interest in recent years. The work presented here demonstrates the antiviral potential of DBD plasma in the treatment of HSV-1 corneal infection. The advantage of plasma technology is its high degree of versatility and adaptability. Non-thermal plasmas can be generated at a wide range of energy settings, in various customized gaseous media, and using a growing variety of electrodes. This multitude of parameters involved in plasma generation allows for fine-tuning of the nature, quality, and intensity of the produced plasma in order to fit a specific biomedical need. Dielectric barrier discharge plasma electrodes can be manufactured in different shapes and sizes, and the use of microelectrodes holds great potential for novel methods of targeted intervention within precise anatomical locations on the ocular surface as well as in the internal structures of the eye.

Preliminary measurements of plasmas produced in the present systems were performed using Fourier transform infrared absorption spectroscopy and show that DBD plasma in atmospheric gas phase generates ozone (1.7×10¹⁷ cm⁻³), H₂O₂ (4.2×10¹⁷ cm⁻³), and N₂O groups (2×10¹⁵ cm⁻³), whereas the main component in liquid is H₂O₂, with generation rate of approximately 4 micro-M/J. However, it is currently unclear what minor species may be generated from the various organic components present in the treated cell culture medium.

The use of DBD plasma-treated liquids may provide useful therapeutic advantages for the treatment of corneal herpetic infections. Since this is a nonpharmacologic agent with a mechanism of action unrelated to the inhibition of HSV-1 DNA polymerase, it may serve as a unique option for patients with drug-resistant infection. It could also be used in combination therapy with established antiviral agents. Such embodiments are considered within the scope of the present invention(s). The common target of the majority of current antiherpetic medications allows for the development of multidrug resistance. This is a growing concern in the immunocompromised population, where suppression of infection relies entirely on therapeutic intervention. Thus, the addition of DBD plasma to the accepted drug armamentarium in these patients may counteract the development of resistance. In addition, recent interest in the use of plasmas for the enhancement of wound healing, including corneal ulceration, could point to a possible 2-fold effect of DBD plasma whereby the reduction of viral load is accompanied by expedited resolution of the epithelial ulcer. Further, DBD plasma may have analogous antiviral activity against other members of the herpesviridae family, which includes such prominent ocular pathogens as varicella zoster virus, herpes simplex virus type 2, Epstein-Barr virus, and cytomegalovirus. Again, such treatments are also considered within the scope of the present invention, including those treatment conditions as described herein.

### Example 3. Stability of Plasma Treated Liquid

Solutions of phosphate buffered saline (Ca/Mg-free)(PBS), PBS+100 mM valine or growth media containing 10% fetal calf serum were treated with micro or nano-second discharge plasma. The liquids designated as treated with “Nano Plasma” were subjected to a non-thermal plasma generated at 15.5 kV and 550 Hz for 16 seconds. The liquids designated as treated with “Micro Plasma” were subjected to a non-thermal plasma generated at about 19 kV and 1800 Hz for 20 seconds.

The media were placed in airtight containers and added to MCF10A cells at the indicated time, (referred to as holding or treatment time). One hour after the addition of media, cell lysates were prepared and subjected to SDS PAGE and Western blot with the indicated antibody. Gamma-H2AX is an indicator of DNA damage and phosph-Chk2pT68 was a measure of ATM and ATR activation. Total Chk2 and nucleolin were controls. The results of these experiments are shown in FIG. 13A. FIG. 13B shows the results of tests conducted under the same experimental conditions, except that the time points are as indicated and pChk2 was not tested.

These experiments showed that PBS treated with nanosecond discharge plasma showed no loss in DNA damaging activity up to 48 hours after treatment. Solutions containing media were less stable with either nano- or microsecond discharge plasma, whereas solutions treated with microsecond discharge plasma were much less stable.

While the embodiments have been described in connection with the preferred embodiments of the various figures, it is to be understood that other similar embodiments may be used or modifications and additions may be made to the described embodiment for performing the same function without deviating therefrom. Therefore, the disclosed embodiments should not be limited to any single embodiment but rather should be construed in breadth and scope in accordance with the appended claims.

As those skilled in the art will appreciate, numerous modifications and variations of the present invention are possible in light of these teachings, and all such are contemplated hereby. For the sake of brevity, each and every combination is not provided here, but the skilled artisan would appreciate that, in addition to the embodiments described herein, the present invention contemplates and claims those inventions resulting from the combination of each and every feature of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, in their entirety, for all purposes.

1. A method of treating herpes keratitis comprising contacting an eye of a patient in need of such treatment with a disinfecting aqueous fluid, the aqueous fluid being previously contacted with a non-thermal plasma.

2. The method of claim 1, wherein the treating reduces the number of HSV-1 genome copies in the eye, relative to the number of HSV-1 genome copies without treatment.

3. The method of claim 1, wherein the treating reduces the number of HSV-1 genome copies in the eye by at least 20%, relative to the number of HSV-1 genome copies before treatment.

4. The method of claim 1, wherein the fluid is a liquid.

5. The method of claim 1, wherein the fluid is a misted or aerosolized liquid.

6. The method of claim 1, wherein the aqueous fluid comprises saline, phosphate buffer, or a combination thereof.

7. The method of claim 1, wherein the non-thermal plasma is derived from a dielectric barrier discharge, a corona or pulsed corona discharge, arc, spark, gliding arc, radio frequency discharge, microwave discharge or any combination thereof.

8. The method of claim 1, wherein the plasma is a non-thermal plasma having an intensity of at least about 0.1 J/cm² at the surface of a plasma source electrode.

9. The method of claim 1, wherein the fluid that has been contacted with the non-thermal plasma for a time in a range of from about 5 seconds to about 5 minutes.

10. The method of claim 1, wherein the irrigating is done within a time in a range of from about one minute to about 10 minutes after the fluid has been contacted with the non-thermal plasma.

11. The method of claim 1, wherein the irrigating is done for a period in a range of from about 5 seconds to about 5 minutes.

12. The method of claim 1, wherein the irrigating is done two or more times.

13. The method of claim 7, wherein the non-thermal plasma is derived from a dielectric barrier discharge.

14. The method of claim 1, wherein the non-thermal plasma is generated using a configuration providing a maximum frequency in a range of from about 0.5 to about 2 kHz.

15. The method of claim 1, wherein the non-thermal plasma is generated using a configuration providing an amplitude in a range of from about 5 to 25 kV.


SEQUENCE LISTING (10 sequences)

SEQ ID NO: 1 - DNA, 17 bp, synthetic construct
  misc_feature 1..17: note = Description of Artificial Sequence: Synthetic primer
  source 1..17: mol_type = other DNA
ctgcgctgcgacacctt

SEQ ID NO: 2 - DNA, 22 bp, synthetic construct
  misc_feature 1..22: note = Description of Artificial Sequence: Synthetic primer
  source 1..22: mol_type = other DNA
caattgcatccaggttttcatg

SEQ ID NO: 3 - DNA, 22 bp, synthetic construct
  misc_feature 1..22: note = Description of Artificial Sequence: Synthetic primer
  source 1..22: mol_type = other DNA
agagggacatccaggactttgt

SEQ ID NO: 4 - DNA, 19 bp, synthetic construct
  misc_feature 1..19: note = Description of Artificial Sequence: Synthetic primer
  source 1..19: mol_type = other DNA
caggcgcttgttggtgtac

SEQ ID NO: 5 - DNA, 20 bp, synthetic construct
  misc_feature 1..20: note = Description of Artificial Sequence: Synthetic primer
  source 1..20: mol_type = other DNA
attccacccgcatggagttc

SEQ ID NO: 6 - DNA, 20 bp, synthetic construct
  misc_feature 1..20: note = Description of Artificial Sequence: Synthetic primer
  source 1..20: mol_type = other DNA
cggtgatgttcgtcaggacc

SEQ ID NO: 7 - DNA, 20 bp, synthetic construct
  misc_feature 1..20: note = Description of Artificial Sequence: Synthetic primer
  source 1..20: mol_type = other DNA
gcttgccctgtccagttaat

SEQ ID NO: 8 - DNA, 20 bp, synthetic construct
  misc_feature 1..20: note = Description of Artificial Sequence: Synthetic primer
  source 1..20: mol_type = other DNA
tagctcagctgcacccttta

SEQ ID NO: 9 - DNA, 20 bp, synthetic construct
  misc_feature 1..20: note = Description of Artificial Sequence: Synthetic primer
  source 1..20: mol_type = other DNA
gtaacccgttgaaccccatt

SEQ ID NO: 10 - DNA, 20 bp, synthetic construct
  misc_feature 1..20: note = Description of Artificial Sequence: Synthetic primer
  source 1..20: mol_type = other DNA
ccatccaatcggtagtagcg